(19) United States
(12) Patent Application Publication
(10) Pub. No.: US 2007/0092506 A1
(43) Pub. Date: Apr. 26, 2007

(54) Use of rapamycin and agents that inhibit B7 activity in immunomodulation

(75) Inventors: Joseph Sypek, Newton, MA (US); Mark J. Collins, Candia, NH (US)

Correspondence Address: FOLEY HOAG, LLP;PATENT GROUP, (w/WYS), 155 SEAPORT BLVD., BOSTON, MA, 02210-2600, US

(73) Assignee: Genetics Institute, LLC, Cambridge, MA (US)

(21) Appl. No.: 11/585,788

(22) Filed: Oct. 24, 2006

Related U.S. Application Data
(63) Continuation of application No. 09/805,800, filed on Mar. 13, 2001, now abandoned.
(60) Provisional application No. 60/189,106, filed on Mar. 14, 2000.

(51) Int. Cl. A61K 39/395; A61K 31/4745

(52) U.S. Cl. 424/133.1; 424/144.1; 514/291

(57) ABSTRACT

The invention provides methods for downmodulating an immune response comprising contacting immune cells from a subject with at least one agent that binds to at least one B7 molecule in combination with a Rapamycin compound.

### RELATED APPLICATION

The present application claims priority to U.S. Provisional Patent Application Ser. No. 60/189,106, filed Mar. 14, 2000, entitled “Use of Anti-B7 Antibodies and Rapamycin in Immunomodulation”, the entire contents of which are expressly incorporated by reference.

### BACKGROUND OF THE INVENTION

In order for T cells to respond to foreign proteins, two signals must be provided by antigen-presenting cells (APCs) to resting T lymphocytes (Jenkins, M. and Schwartz, R. (1987) J. Exp. Med. 165, 302-319; Mueller, D. L., et al. (1990) J. Immunol. 144, 3701-3709). The first signal, which confers specificity to the immune response, is transduced via the T cell receptor (TCR) following recognition of foreign antigenic peptide presented in the context of the major histocompatibility complex (MHC). The second signal, termed costimulation, induces T cells to proliferate and become functional (Lenschow et al. 1996. Annu. Rev. Immunol. 14:233). Costimulation is neither antigen-specific, nor MHC restricted and is thought to be provided by one or more distinct cell surface molecules expressed by APCs (Jenkins, M. K., et al. 1988 J. Immunol. 140, 3324-3330; Linsley, P. S., et al. 1991 J. Exp. Med. 173, 721-730; Gimmi, C. D., et al., 1991 Proc. Natl. Acad. Sci. USA. 88, 6575-6579; Young, J. W., et al. 1992 J. Clin. Invest. 90, 229-237; Koulova, L., et al. 1991 J. Exp. Med. 173, 759-762; Reiser, H., et al. 1992 Proc. Natl. Acad. Sci. USA. 89, 271-275; van-Seventer, G. A., et al. (1990) J. Immunol. 144, 4579-4586; LaSalle, J. M., et al., 1991 J. Immunol. 147, 774-80; Dustin, M. I., et al., 1989 J. Exp. Med. 169, 503; Armitage, R. J., et al. 1992 Nature 357, 80-82; Liu, Y., et al. 1992 J. Exp. Med. 175, 437-445).

The CD80 (B7-1) and CD86 (B7) proteins, expressed on APCs, are critical costimulatory molecules (Freeman et al. 1991. J. Exp. Med. 174:625; Freeman et al. 1989 J. Immunol. 143:2714; Azuma et al. 1993 Nature 366:76; Freeman et al. 1993. Science 262:909). B7 appears to play a predominant role during primary immune responses, while B7-1, which is upregulated later in the course of an immune response, may be important in prolonging primary T cell responses or costimulating secondary T cell responses (Bluestone. 1995. Immunity. 2:555).

One receptor to which B7-1 and B7 bind, CD28, is constitutively expressed on resting T cells and increases in expression after activation. After signaling through the T cell receptor, ligation of CD28 and transduction of a costimulatory signal induces T cells to proliferate and secrete IL-2 (Linsley, P. S., et al. 1991 J. Exp. Med. 173, 721-730; Gimmi, C. D., et al. 1991 Proc. Natl. Acad. Sci. USA. 88, 6575-6579; June, C. H., et al. 1990 Immunol. Today. 11, 211-6; Harding, F. A., et al. 1992 Nature. 356, 607-609). A second receptor, termed CTLA4 (CD152) is homologous to CD28 but is not expressed on resting T cells and appears following T cell activation (Brunet, J. F., et al., 1987 Nature 328, 267-270). CTLA4 appears to be critical in negative regulation of T cell responses (Waterhouse et al. 1995. Science 270:985). Blockade of CTLA4 has been found to remove inhibitory signals, while aggregation of CTLA4 has been found to provide inhibitory signals that downregulate T cell responses (Allison and Krummel. 1995. Science 270:932). The B7 molecules have a higher affinity for CTLA4 than for CD28 (Linsley, P. S., et al., 1991 J. Exp. Med. 174, 561-569) and B7-1 and B7 have been found to bind to distinct regions of the CTLA4 molecule and have different kinetics of binding to CTLA4 (Linsley et al. 1994. Immunity. 1:793). A new molecule related to CD28 and CTLA4, ICOS, has been identified and seems to be important in IL-10 production (Hutloff et al. 1999. Nature. 397:263; WO 98/38216). If T cells are only stimulated through the T cell receptor, without receiving an additional costimulatory signal, they become nonresponsive, anergic, or die, resulting in downmodulation of the immune response.

A new molecule related to CD28 and CTLA4, ICOS, has been identified (Hutloff et al. (1999) Nature 397:263; WO 98/38216; Tamatani, T. et al. (2000) Int. Immunol. 12:51-55), as has its ligand, GL50 (also called by the names ICOSL, B7h, LICOS, and B7RP-1) which is a new B7 family member (Ling, V. et al. (2000) J. Immunol. 164:1653-7; Swallow, M. M. et al. (1999) Immunity 11:423-432; Aicher, A. et al. (2000) J. Immunol. 164:4689-96; Mages, H. W. et al. (2000) Eur. J. Immunol. 30:1040-7; Brodie, D. et al. (2000) Curr. Biol. 10:333-6; Yoshinaga, S. K. et al. (1999) Nature 402:827-32). An additional B7 family member, B7-H1, has also been identified (Dong, H. et al. (1999) Nat. Med. 5:1365-1369). B7-H1, also known as PD-L1, interacts with the immunoinhibitory receptor PD-1 (Freeman, G. J. et al. (2000) J. Exp. Med. 192:1027-34).

The importance of the B7:CD28/CTLA4 costimulatory pathway has been demonstrated in vitro and in several in vivo model systems. Blockade of this costimulatory pathway results in the development of antigen specific tolerance in murine and humans systems (Harding, F. A., et al. (1992) Nature. 356, 607-609; Lenschow, D. J., et al. (1992) Science. 257, 789-792; Turka, L. A., et al. (1992) Proc. Natl. Acad. Sci. USA. 89, 11102-11105; Gimmi, C. D., et al. (1993) Proc. Natl. Acad. Sci USA 90, 6586-6590; Boussiotis, V., et al. (1993) J. Exp. Med. 178, 1753-1763). Conversely, expression of B7 by B7 negative murine tumor cells induces T-cell mediated specific immunity accompanied by tumor rejection and long lasting protection to tumor challenge (Chen, L., et al. (1992) Cell 71, 1093-1102; Townsend, S. E. and Allison, J. P. (1993) Science 259, 368-370; Baskar, S., et al. (1993) Proc. Natl. Acad. Sci. 90, 5687-5690.). Therefore, manipulation of the costimulatory pathways offers great potential to stimulate or suppress immune responses in humans.

### SUMMARY OF THE INVENTION

In one aspect, the invention provides a method for downmodulating an immune response comprising contacting immune cells from a subject with at least one antibody that binds to at least one B7 molecule in combination with a Rapamycin compound.

In one embodiment, the cells are contacted with two antibodies that bind at least two different B7 molecules.

In another embodiment, the step of contacting is performed ex vivo. In another embodiment, the step of contacting is performed in vivo.

In another aspect, the invention provides a method for downmodulating an immune response in a subject having an autoimmune disorder comprising contacting immune cells from the subject with at least one antibody that binds to at least one B7 molecule in combination with a Rapamycin compound.

In one embodiment, the immune cells are contacted with two antibodies that bind at least two different B7 molecules.

In one embodiment, the autoimmune disorder is systemic lupus erythematosis.

In another aspect, the invention provides a method for downmodulating an immune response in a subject suffering from systemic lupus erythematosis comprising administering to the subject an antibody that binds to B7-1, an antibody that binds to B7-2, and a Rapamycin compound, wherein the antibody that binds to B7-1 and the antibody that binds to B7-2 are administered over at least one short course of therapy.

In yet another aspect, the invention provides a method for downmodulating an immune response in a subject suffering from systemic lupus erythematosis comprising administering to the subject an antibody that binds to B7-1, an antibody that binds to B7-2, and a Rapamycin compound, wherein the Rapamycin compound is administered over at least one intermediate course of therapy.

### FIGURES

FIG. 1 shows the average proteinuria grade in NZB/NZW F1 female mice with no treatment or combination treatment with anti-B7.1 and anti-B7.2. Proteinuria grading is as follows: Grade 0.5 is “trace” proteinuria; Grade 1 equals ˜30 mg/dL; Grade 2 equals ˜100 mg/dL; Grade 3 equals ˜300 mg/dL; Grade 4 equals >2000 mg/dL; and Grade 5 represents death. Clinically significant levels of proteinuria is shown by the dotted line.

FIG. 2 is a table illustrating the dosing protocol used to treat NZB/NZW F1 (B/W) mice. In particular, the amounts of anti-B7.1, anti-B7.2, and Rapamycin are highlighted as well as the duration and timing of the therapy.

FIG. 3 is a table illustrating the histopathologic Evaluation of NZB×NZW F1 hybrid female mice at 42 weeks of age after no therapy or treatment with Rapamycin 8 weeks (weeks 29-36). Histology was graded according to a predetermined arbitrary scale as follows: Normal: 0; Slight=1; Mild=2; Moderate=3; Marked=4; Severe=5; and Focal not Diffuse=( ).

FIG. 4(A) shows survival curves for NZB/NZW F1 (B/W) mice after no treatment, single therapy treatment, early versus late dosing treatment, or combination therapy treatment. Early Rapamycin treatment began at 25 weeks and the late Rapamycin treatment began at 33 weeks. Each group contained ten mice. (B) shows average proteinuria grade for mice subjected to no treatment, single therapy treatment, early versus late Rapamycin treatment, or combination therapy treatment. Average proteinuria was graded as described in FIG. 1. Clinically significant levels of proteinuria is shown by the dotted line.

FIG. 5 shows (A) Anti-nDNA antibody isotypes as measured by ELISA after single therapeutic dosing treatment; (B) Anti-nDNA antibody isotypes measured by ELISA after combined therapeutic dosing treatment. For both treatments, serum immunoglobulins are classified as either IgG₁ or IgG₂ₐ isotypes.

### DETAILED DESCRIPTION OF THE INVENTION

The instant invention is based, at least in part, on the finding that agents that decrease co-stimulatory signals to T cells are more efficient in reducing symptoms of autoimmune disease when used in combination with Rapamycin or Rapamycin-like compounds. The instant invention provides improved methods of downmodulating immune responses by a cell expressing a B7 molecule with a combination of at least one antibody which binds to at least one B7 molecule and a Rapamycin compound. In a preferred embodiment, at least two antibodies which bind to at least two different B7 molecules are contacted with a cell expressing a B7 molecule in combination with a Rapamycin compound. The subject methods are useful for downmodulation of unwanted immune responses, e.g., autoimmune responses. The subject methods can be performed either in vitro or in vivo. Preferably, the methods of the invention are used to treat systemic lupus erythematosis

Before further description of the invention, certain terms employed in the specification, examples and appended claims are, for convenience, collected here.

Definitions

As used herein, the term “combination therapy” includes a combination of at least one antibody that binds at least one B7 molecule and a Rapamycin compound. For example, an antibody that binds to B7-1 or antibody that binds to B7-2 can be used in a combination with a Rapamycin compound in a combination therapy. At least one antibody that binds a B7 molecule and a Rapamycin compound need not be administered at the same time or at the same site of administration. Preferably two antibodies that bind to two different B7 molecules are administered in combination with a Rapamycin compound.

As used herein, the term “immune cell” includes cells that are of hematopoietic origin and that play a role in the immune response. Immune cells include lymphocytes, such as B cells and T cells; natural killer cells and myeloid cells, such as monocytes, macrophages, eosinophils, mast cells, basophils, and granulocytes.

As used herein, the term “immune response” includes T and/or B cell responses, i.e., cellular and/or humoral immune responses. In one embodiment, the claimed methods can be used to reduce T helper cell responses. In another embodiment, the claimed methods can be used to reduce cytotoxic T cell responses. The claimed methods can be used to reduce both primary and secondary immune responses. The immune response of a subject can be determined by, for example, assaying antibody production, immune cell proliferation, the release of cytokines, the expression of cell surface markers, cytotoxicity, etc.

As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal can result in cytokine secretion, e.g., in a T cell that has received a T cell-receptor-mediated signal. As used herein the term “costimulatory molecule” includes molecules which are present on antigen presenting cells (e.g., B7-1, B7, B7RP-1 (Yoshinaga et al. 1999. Nature 402:827), B7h (Swallow et al. 1999. Immunity. 11:423) and/or related molecules (e.g., homologs)) that bind to costimulatory receptors (e.g., CD28, CTLA4, ICOS (Hutloff et al. 1999. Nature 397:263), B7h ligand (Swallow et al. 1999. Immunity. 11:423) and/or related molecules) on T cells. These molecules are also collectively referred to herein as “B7 molecules.”

As used herein, the language “B7” or “B7 molecule” includes naturally occurring B7-1 molecules, B7-2 molecules, B7RP-1 molecules (Yoshinaga et al. 1999. Nature 402:827), B7h molecules (Swallow et al. 1999. Immunity. 11:423), structurally related molecules, fragments of such molecules, and/or functional equivalents thereof. The term “equivalent” is intended to include amino acid sequences encoding functionally equivalent costimulatory molecules having an activity of a B7 molecule, e.g., the ability to bind to the natural ligand(s) of B7 on immune cells, such as CTLA4, ICOS, and/or CD28 on T cells, and the ability to modulate immune cell costimulation.

As used here, the term “agent that blocks a B7 activity” includes those agents that interfere with the ability of a B7 molecule to bind its natural ligand and/ or that interfere with the ability of a B7 molecule to costimulate T cells, e.g., as measured by cytokine production and/or proliferation. Exemplary agents include blocking antibodies, peptides that block the ability of B7 to bind to its natural ligand but which fail to transmit a costimulatory signal to a T cell, peptidomimetics, small molecules, and the like.

The term “antibody”, as used herein, includes immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The phrase “complementary determining region” (CDR) includes the region of an antibody molecule which comprises the antigen binding site.

The antibody may be an IgG such as IgG1, IgG2, IgG3 or IgG4; or IgM, IgA, IgE or IgD isotype. The constant domain of the antibody heavy chain may be selected depending upon the effector function desired. The light chain constant domain may be a kappa or lambda constant domain.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., the extracellular domain of a B7 molecule). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

Antibodies may be polyclonal or monoclonal; xenogeneic, allogeneic, or syngeneic; or modified forms thereof, e.g. humanized, chimeric, etc. Preferably, antibodies of the invention bind specifically or substantially specifically to B7-targeted molecules. The terms “monoclonal antibodies” and “monoclonal antibody composition”, as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody composition, typically displays a single binding affinity for a particular antigen with which it immunoreacts.

The antibodies described herein may be humanized. The term “humanized antibody”, as used herein, includes antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

An “isolated antibody”, as used herein, includes an antibody that is substantially free of other antibodies having different antigenic specificities (e.g., an isolated antibody that is substantially free of antibodies that specifically bind antigens other than the protein the antibody is directed against). Moreover, an isolated antibody may be substantially free of other cellular material and/or chemicals.

As used herein, the term “extracellular domain of a B7 molecule” includes a portion of a B7 molecule which, in the cell-associated form of a B7 molecule, is extracellular. A B7 extracellular domain includes the portion of a B7 molecule which mediates binding to a costimulatory receptor, e.g., CD28, ICOS, and/or CTLA4. For example, the human B7-1 extracellular domain comprises from about amino acid 1 to about amino acid 208 and the human B7 extracellular domain comprises from about amino acid 24 to about amino acid 245. See, for example, B7-2 (Freeman et al. 1993 Science. 262:909; GenBank Accession numbers P42081 or A48754; or U.S. Pat. No. 5,942,607); B7-1 (Freeman et al. J. Exp. Med. 1991. 174:625; GenBank Accession numbers P33681 or A45803; or U.S. Pat. No. 5,858,776).

The language “a desired binding specificity for a B7 epitope”, as well as the more general language “an antigen binding site which specifically binds (immunoreacts with)”, refers to the ability of individual antibodies to specifically immunoreact with a peptide having a B7 costimulatory activity. That is, it refers to a non-random binding reaction between an antibody molecule and an antigenic determinant of B7. Illustrative of a specific antibody-antigen complex is that between antibody 2D10 and mouse B7-2 (J Immunol 1994 152: 2105-14). The desired binding specificity is typically determined from the reference point of the ability of the antibody to differentially bind a B7 antigen and an unrelated antigen, and therefore distinguish between two different antigens -particularly where the two antigens have unique epitopes. In other embodiments, the desired binding affinity refers to the ability of the antibody to discriminate in binding between different isoforms of B7 antigens or between different B7 antigens. An antibody which binds specifically to a B7 epitope is referred to as a “specific antibody”.

Preferably, the anti-B7 antibodies of the invention bind to naturally occurring forms of B7, but are substantially unreactive, e.g., have background binding to unrelated, non-B7 molecules. Antibodies specific for a B7 molecule from one source, e.g., human B7-1 may or may not be reactive with B7-1 molecules from different species. In addition, antibodies specific for naturally occurring B7 molecules may or may not bind to mutant forms of such molecules. In one embodiment, mutations in the amino acid sequence of a naturally occurring B7 molecule result in modulation of the binding (e.g., either increased or decreased binding) of the antibody to the B7 molecule. Antibodies to B7 molecules can be readily screened for their ability to meet this criteria. Assays to determine affinity and specificity of binding are known in the art, including competitive and non-competitive assays. Assays of interest include ELISA, RIA, flow cytometry, etc. Binding assays may use purified or semi-purified B7 protein, or alternatively may use cells that express B7, e.g. cells transfected with an expression construct for B7. As an example of a binding assay, purified B7 protein is bound to an insoluble support, e.g. microtiter plate, magnetic beads, etc. The candidate antibody and soluble, labeled CTLA4 or CD28 are added to the cells, and the unbound components are then washed off. The ability of the antibody to compete with CTLA4 or CD28 for B7 binding is determined by quantitation of bound, labeled CTLA4 or CD28. An isolated antibody that specifically binds human B7 may, however, have cross-reactivity to other antigens, such as B7 molecules from other species.

“Antibody combining site”, as used herein, refers to that structural portion of an antibody molecule comprised of a heavy and light chain variable and hypervariable regions that specifically binds (immunoreacts with) antigen. The term “immunoreact” or “reactive with” in its various forms is used herein to refer to binding between an antigenic determinant-containing molecule and a molecule containing an antibody combining site such as a whole antibody molecule or a portion thereof.

The term “antigenic determinant”, as used herein, refers to the actual structural portion of the antigen that is immunologically bound by an antibody combining site. The term is also used interchangeably with “epitope”.

As used herein the language “short course of therapy” includes a therapeutic regimen that is of relatively short duration relative to the course of the illness being treated. For example a short course of therapy may last between about one to about four weeks. In contrast, “an intermediate course of therapy” includes a therapeutic regimen that is of longer duration than a short course of therapy. For example, an intermediate course of therapy can last from more than one month to about four months (e.g., between about five to about 16 weeks). An “extended course of therapy” includes those therapeutic regimens that last longer than about four months, e.g., from about five months on. For example, an extended course of therapy may last from about six months to as long as the illness persists. The appropriateness of one or more of the courses of therapy described above for any one individual can readily be determined by one of ordinary skill in the art. In addition, the treatment appropriate for a subject may be changed over time as required.

In the course of therapy, doses can be administered early or late. As used herein, “early dosing” includes a therapeutic regimen where Rapamycin is administered to a patient at the onset of disease, e.g. at the onset of clinical symptoms. For example, in the case of SLE, at the onset of proteinuria. Alternatively, “late dosing” includes a therapeutic regimen where Rapamycin is administered to a patient some time after the onset of clinical symptoms, e.g. in the case of SLE, after the onset of renal disease.

It will also be understood that one or more anti-B7 antibodies can be administered using a different course of therapy than is used to administer a Rapamycin compound. For example an anti-B7 antibody can be administered using a short course of therapy during an induction phase of an immune response, e.g., during flare periods of lupus, and a Rapamycin compound may be administered over an intermediate course later the induction phase of the immune response.

II. B7 Molecules and Agents that Block B7 Activity

The B7 antigens are a family of costimulatory molecules found on the surface of B lymphocytes, professional antigen presenting cells (e.g., monocytes, dendritic cells, Langerhan cells) and cells which present antigen to immune cells (e.g., keratinocytes, endothelial cells, astrocytes, fibroblasts, oligodendrocytes). These costimulatory molecules bind either CTLA4, CD28, and/or ICOS on the surface of T cells or other known or as yet undefined receptors on immune cells. The members of this family of costimulatory molecules are capable of providing costimulation to activated T cells to thereby induce T cell proliferation and/or cytokine secretion.

Agents that block an activity of a B7 molecule can be derived using B7 nucleic acid or amino acid sequences. For example, nucleotide sequences of costimulatory molecules are known in the art and can be found in the literature or on a database such as GenBank. See, for example, B7-2 (Freeman et al. 1993 Science. 262:909 or GenBank Accession numbers P42081 or A48754); B7-1 (Freeman et al. J. Exp. Med. 1991. 174:625 or GenBank Accession numbers P33681 or A45803; CTLA4 (See e.g., Ginsberg et al. 1985. Science. 228:1401; or GenBank Accession numbers P16410 or 291929); and CD28 (Aruffo and Seed. Proc Natl. Acad. Sci. 84:8573 or GenBank Accession number 180091), ICOS (Hutloffet al. 1999. Nature. 397:263; WO 98/38216), PD-1 (Ishida et al. (1992) EMBO J. 11:3887; Shinohara et al. (1994) Genomics 23:704) and related sequences. Purification techniques for B7 molecules have been established, and, additionally, B7 genes (cDNA) have been cloned from a number of species, including human and mouse (see, for example, Freeman, G. J. et al. (1993) Science 262:909-911; Azuma, M. et al. (1993) Nature 366:76-79; Freeman, G. J. et al. (1993) J. Exp. Med. 178:2185-2192).

Purification techniques for B7 molecules have been established, and, additionally, B7 genes (cDNA) have been cloned from a number of species, including human and mouse (see, for example, Freeman, G. J. et al. (1993) Science 262:909-911; Azuma, M. et al. (1993) Nature 366:76-79; Freeman, G. J. et al. (1993) J. Exp. Med. 178:2185-2192).

Nucleotide sequences of costimulatory molecules are known in the art and can be found in the literature or on a database such as GenBank. See, for example, B7-2 (Freeman et al. 1993 Science. 262:909 or GenBank Accession numbers P42081 or A48754); B7-1 (Freeman et al. J. Exp. Med. 1991. 174:625 or GenBank Accession numbers P33681 or A45803; CTLA4 (See e.g., Ginsberg et al. 1985. Science. 228:1401; or GenBank Accession numbers P16410 or 291929); and CD28 (Aruffo and Seed. Proc Natl. Acad. Sci. 84:8573 or GenBank Accession number 180091), ICOS (Hutloff et al. 1999. Nature. 397:263; WO 98/38216), and related sequences.

In addition to naturally occurring forms of costimulatory molecules, the term “costimulatory molecule” also includes non-naturally occurring forms, e.g., mutant forms of costimulatory molecules which retain the function of a costimulatory molecule, e.g., the ability to bind to cognate counter receptor. For example, DNA sequences capable of hybridizing to DNA encoding a B7 molecule, under conditions that avoid hybridization to non-costimulatory molecule genes, (e.g., under conditions equivalent to 65° C. in 5×SSC (1×SSC=150 mM NaCl/0.15 M Na citrate)) can be used to make antiB7 antibodies. Alternatively, DNA sequences which retain sequence identity over regions of the nucleic acid molecule which encode protein domains which are important in costimulatory molecule function, e.g., binding to other costimultory molecules, can be used to produce costimulatory proteins which can be used as immunogens. Preferably, nonnaturally occurring costimulatory molecules have significant (e.g., greater than 70%, preferably greater than 80%, and more preferably greater than 90-95%) amino acid identity with a naturally occurring amino acid sequence of a costimulatory molecule extracellular domain.

To determine amino acid residues of a costimulatory molecule which are likely to be important in the binding of a costimulatory molecule to its counter receptor, amino acid sequences comprising the extracellular domains of costimulatory molecules of different species, e.g., mouse and human, can be aligned and conserved (e.g., identical) residues noted. This can be done, for example, using any standard alignment program, such as MegAlign (DNA STAR). Such conserved or identical residues are likely to be necessary for proper binding of costimulatory molecules to their receptors and are, thus, not likely to be amenable to alteration.

For example, the regions of the B7-1 molecule which are important in mediating the functional interaction with CD28 and CTLA4 have been identified by mutation. Two hydrophobic residues in the V-like domain of B7-1, including the Y87 residue, which is conserved in all B7-1 and B7-2 molecules cloned from various species, were found to be critical (Fargeas et al. 1995. J. Exp. Med. 182:667). Using these, or similar, techniques amino acid residues of the extracellular domains of costimulatory molecules which are critical and, therefore, not amenable to alteration can be determined.

Using B7 cDNA molecules, peptides having an activity of B7 can be produced using standard techniques. Host cells transfected to express peptides can be any procaryotic or eucaryotic cell. For example, a peptide having B7 activity can be expressed in bacterial cells such as E. coli, insect cells (baculovirus), yeast, or mammalian cells such as Chinese hamster ovary cells (CHO) and NS0 cells. Other suitable host cells and expression vectors may be found in Goeddel, (1990) supra or are known to those skilled in the art. Examples of vectors for expression in yeast S. cerivisae include pYepSec1 (Baldari. et al., (1987) Embo J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Baculovirus vectors available for expression of proteins in cultured insect cells (SF 9 cells) include the pAc series (Smith et al., (1983) Mol. Cell Biol. 3:2156-2165) and the pVL series (Lucklow, V. A., and Summers, M. D., (1989) Virology 170:31-39). Generally, COS cells (Gluzman, Y., (1981) Cell 23:175-182) are used in conjunction with such vectors as pCDM8 (Seed, B., (1987) Nature 329:840) for transient amplification/expression in mammalian cells, while CHO (dhfr-Chinese Hamster Ovary) cells are used with vectors such as pMT2PC (Kaufman et al. (1987), EMBO J. 6:187-195) for stable amplification/expression in mammalian cells. A preferred cell line for production of recombinant protein is the NS0 myeloma cell line available from the ECACC (catalog #85110503) and described in Galfre, G. and Milstein, C. ((1981) Methods in Enzymology 73(13):346; and Preparation of Monoclonal Antibodies: Strategies and Procedures, Academic Press, N.Y., N.Y). Vector DNA can be introduced into mammalian cells via conventional techniques such as calcium phosphate or calcium chloride co-precipitation, DEAE-dextran-mediated transfection, lipofectin, or electroporation. Suitable methods for transforming host cells can be found in Sambrook et al. (Molecular Cloning: A Laboratory Manual, 2nd Edition, Cold Spring Harbor Laboratory press (1989)), and other laboratory textbooks. When used in mammalian cells, the expression vector's control functions are often provided by viral material. For example, commonly used promoters are derived from polyoma, Adenovirus 2, cytomegalovirus and most frequently, Simian Virus 40.

Peptides having an activity of B7 expressed in mammalian cells or otherwise can be purified according to standard procedures of the art, including ammonium sulfate precipitation, fractionation column chromatography (e.g. ion exchange, gel filtration, electrophoresis, affinity chromatography, etc.) and ultimately, crystallization (see generally, “Enzyme Purification and Related Techniques”, Methods in Enzymology, 22:233-577 (1971)).

The present invention also pertains to variants of the B7 polypeptides which function as B7 antagonists. Variants of the B7 polypeptides can be generated by mutagenesis, e.g., discrete point mutation or truncation of a B7 polypeptide. An agonist of the B7 polypeptide can retain substantially the same, or a subset, of the biological activities of the naturally occurring form of a B7 polypeptide. An antagonist of a B7 polypeptide can inhibit one or more of the activities of the naturally occurring form of the B7 polypeptide by, for example, competitively modulating a cellular activity of a B7 polypeptide. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the B7 polypeptide.

In one embodiment, variants of a B7 polypeptide which function as either B7 antagonists can be identified by screening combinatorial libraries of mutants, e.g., truncation mutants, of a B7 (or B7 ligand) polypeptide for B7 antagonist activity. In one embodiment, a variegated library of B7 variants is generated by combinatorial mutagenesis at the nucleic acid level and is encoded by a variegated gene library. A variegated library of B7 variants can be produced by, for example, enzymatically ligating a mixture of synthetic oligonucleotides into gene sequences such that a degenerate set of potential B7 or B7 ligand sequences is expressible as individual polypeptides, or alternatively, as a set of larger fusion proteins (e.g., for phage display) containing the set of B7 or B7 ligand sequences therein. There are a variety of methods which can be used to produce libraries of potential B7 or B7 ligand variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows f6r the provision, in one mixture, of all of the sequences encoding the desired set of potential B7 or B7 ligand sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a B7 or B7 ligand coding sequence can be used to generate a variegated population of B7 or B7 ligand fragments for screening and subsequent selection of variants of a B7 or B7 ligand polypeptide. In one embodiment, a library of coding sequence fragments can be generated by treating a double stranded PCR fragment of a B7 or B7 ligand coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the B7 or B7 ligand.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of B7 or B7 ligand proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify B7 or B7 ligand variants (Arkin and Youvan (1992) Proc. Natl. Acad. Sci. USA 89:7811-7815; Delagrave et al. (1993) Protein Eng. 6(3):327-331).

In one embodiment, cell based assays can be exploited to analyze a variegated B7 or B7 ligand library. For example, a library of expression vectors can be transfected into a cell line which ordinarily synthesizes B7 or B7 ligand. The transfected cells are then cultured such that B7 or B7 ligand and a particular mutant B7 or B7 ligand are secreted and the effect of expression of the mutant on B7 or B7 ligand activity can be detected, e.g., by any of a number of functional assays. DNA can then be recovered from the cells which score for inhibition of B7 or B7 ligand activity, and the individual clones further characterized.

In addition to B7 or B7 ligand polypeptides consisting only of naturally-occurring amino acids, B7 or B7 ligand peptidomimetics are also provided. Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15:29; Veber and Freidinger (1985) TINS p. 392; and Evans et al. (1987) J. Med. Chem. 30:1229, which are incorporated herein by reference) and are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), such as human B7 or B7 ligand, but have one or more peptide linkages optionally replaced by a linkage selected from the group consisting of: —CH2NH—, —CH2S—, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivitization (e.g., labeling) of peptidomimetics should not substantially interfere with the desired biological or pharmacological activity of the peptidomimetic.

Systematic substitution of one or more amino acids of a B7 or B7 ligands amino acid sequence with a D-amino acid of the same type (e.g., D-lysine in place of L-lysine) can be used to generate more stable peptides. In addition, constrained peptides comprising a B7 or B7 ligand amino acid sequence or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide. The amino acid sequences of B7 or B7 ligand polypeptides identified herein will enable those of skill in the art to produce polypeptides corresponding to B7 or B7 ligand peptide sequences and sequence variants thereof. Such polypeptides can be produced in prokaryotic or eukaryotic host cells by expression of polynucleotides encoding a B7 or B7 ligand peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis, and used e.g., as agonists or antagonists of a B7/B7 ligand interaction. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides can be used therapeutically to treat disease, e.g., by altering costimulation in a patient. Peptidomimetics can be made as described, e.g., in WO 98/56401.

An isolated B7 or B7 ligand protein, or a portion or fragment thereof (or a nucleic acid molecule encoding such a polypeptide), can be used as an immunogen can also be used to make an antibody that blocks a B7 activity. In one embodiment, antibodies for use in the instant methods bind to at least one B7 molecule. In yet another embodiment, an antibody of the invention binds to only one B7 molecule (e.g., to B7-1 and not to B7-2). Such antibodies are known in the art. For example, The 2D10 hybridoma, producing the 2D10 antibody, has been described (Journal of Immunology. 1994. 152:2105). In addition, for use in combination with an anti-B7-2 antibody, several anti-B7-1 antibodies are known or are readily available (see, e.g., U.S. Pat. No. 5,869,050; Powers G. D., et al. (1994) Cell. Immunol. 153, 298-311; Freedman, A. S. et al. (1987) J. Immunol. 137:3260-3267; Freeman, G. J. et al. (1989) J. Immunol. 143:2714-2722; Freeman, G. J. et al. (1991) J. Exp. Med. 174:625-631; Freeman, G. J. et al. (1993) Science 262:909-911; WO 96/40915). Such antibodies are also commercially available, e.g., from R&D Systems (Minneapolis, Minn.) and from Research Diagnostics (Flanders, N.J.)

Moreover, it will be appreciated by those skilled in the art that it is within their skill to generate additional agents and screen for their activity by following standard techniques. For instance, B7 molecules from a variety of species, whether in soluble form or membrane bound, can be used to induce the formation of anti-B7 antibodies. Such antibodies may either be polyclonal or monoclonal, or antigen binding fragments of such antibodies. Of particular significance for use in therapeutic applications are antibodies that inhibit binding of B7 with its natural ligand(s) on the surface of immune cells, thereby inhibiting costimulation of the immune cell through the B7-ligand interaction. Preferred anti-B7 antibodies are those capable of inhibiting or downregulating T cell mediated immune responses by binding B7 on the surface of B lymphocytes and preventing interaction of B7 with CTLA4 and/or CD28. Preferably, the combination of antibodies chosen for use in the invention results in increased inhibition of costimulation of an immune cell, such as a T cell, through the B7-ligand interaction, relative to either antibody alone.

Any agent which binds to a B7 moleclue(s) may be used in the subject methods and compositions. In one embodiment, antibodies for use in the instant methods bind to at least one B7 molecule. In yet another embodiment, an antibody of the invention binds to only one B7 molecule (e.g., to B7-1 and not to B7-2). Such antibodies are known in the art. For example, The 2D10 hybridoma, producing the 2D10 antibody, has been described (Journal of Immunology. 1994. 152:2105). In addition, for use in combination with an anti-B7-2 antibody, several anti-B7-1 antibodies are known or are readily available (see, e.g., U.S. Pat. No. 5,869,050). For example, an anti-mouse B7-1 antibody 1G10 has been described (Powers G. D., et al. (1994) Cell. Immunol. 153, 298-311) and an anti-human B7-1 antibody 133 is also available (see Freedman, A. S. et al. (1987) J. Immunol. 137:3260-3267; Freeman, G. J. et al. (1989) J. Immunol. 143:2714-2722; Freeman, G. J. et al. (1991) J. Exp. Med. 174:625-631; Freeman, G. J. et al. (1993) Science 262:909-911).

Moreover, it will be appreciated by those skilled in the art that it is within their skill to generate additional antibodies by following standard techniques. For instance, B7 molecules from a variety of species, whether in soluble form or membrane bound, can be used to induce the formation of yet further anti-B7 antibodies. Such antibodies may either be polyclonal or monoclonal, or antigen binding fragments of such antibodies. Of particular significance for use in therapeutic applications are antibodies that inhibit binding of B7 with its natural ligand(s) on the surface of immune cells, thereby inhibiting costimulation of the immune cell through the B7-ligand interaction. Preferred anti-B7 antibodies are those capable of inhibiting or downregulating T cell mediated immune responses by binding B7 on the surface of B lymphocytes and preventing interaction of B7 with CTLA4 and/or CD28. Preferably, the combination of antibodies chosen for use in the invention results in increased inhibition of costimulation of an immune cell, such as a T cell, through the B7-ligand interaction, relative to either antibody alone.

A. The Immunogen. The term “immunogen” is used herein to describe a composition containing a peptide having an activity of a B7 molecule as an active ingredient used for the preparation of antibodies against a B7 molecule. When a peptide having a B7 molecule activity is used to induce antibodies it is to be understood that the peptide can be used alone, or linked to a carrier as a conjugate, or as a peptide polymer.

Peptides having an activity of a B7 molecule expressed in mammalian cells or otherwise can be purified according to standard procedures of the art, including ammonium sulfate precipitation, fractionation column chromatography (e.g. ion exchange, gel filtration, electrophoresis, affinity chromatography, etc.) and ultimately, crystallization (see generally, “Enzyme Purification and Related Techniques”, Methods in Enzymology, 22:233-577 (1971)).

To generate suitable anti-B7 molecule antibodies, the immunogen should contain an effective, immunogenic amount of a peptide having a B7 molecule activity, typically as a conjugate linked to a carrier. The effective amount of peptide per unit dose depends, among other things, on the species of animal inoculated, the body weight of the animal and the chosen immunization regimen as is well known in the art. The immunogen preparation will typically contain peptide concentrations of about 10 micrograms to about 500 milligrams per immunization dose, preferably about 50 micrograms to about 50 milligrams per dose. An immunization preparation can also include an adjuvant as part of the diluent. Adjuvants such as complete Freund's adjuvant (CFA), incomplete Freund's adjuvant (IFA) and alum are materials well known in the art, and are available commercially from several sources.

Those skilled in the art will appreciate that, instead of using naturally occurring forms of a B7 molecule for immunization, synthetic peptides can alternatively be employed towards which antibodies can be raised for use this invention. Both soluble and membrane bound costimulatory molecule or peptide fragments are suitable for use as an immunogen and can also be isolated by immunoaffinity purification as well. A purified form of a B7 molecule protein, such as may be isolated as described above or as known in the art, can itself be directly used as an immunogen, or alternatively, can be linked to a suitable carrier protein by conventional techniques, including by chemical coupling means as well as by genetic engineering using a cloned gene of the a costimulatory molecule.

The peptide or protein chosen for immunization can be modified to increase its immunogenicity. For example, techniques for conferring immunogenicity on a peptide include conjugation to carriers or other techniques well known in the art. Any peptide chosen for immunization can also be synthesized. In certain embodiments, such peptides can be synthesized as branched polypeptides, to enhance immune responses, as is known in the art (see, e.g., Peptides. Edited by Bernd Gutte Academic Press 1995. pp. 456-493).

The purified B7 molecule protein can also be covalently or noncovalently modified with non-proteinaceous materials such as lipids or carbohydrates to enhance immunogenecity or solubility. Alternatively, a purified B7 molecule protein can be coupled with or incorporated into a viral particle, a replicating virus, or other microorganism in order to enhance immunogenicity. The B7 molecule protein may be, for example, chemically attached to the viral particle or microorganism or an immunogenic portion thereof.

In an illustrative embodiment, a purified B7 molecule protein, or a peptide fragment having a B7 molecule activity (e.g., produced by limited proteolysis or recombinant DNA techniques) is conjugated to a carrier which is immunogenic in animals. Preferred carriers include proteins such as albumin, serum proteins (e.g., globulins and lipoproteins), and polyamino acids. Examples of useful proteins include bovine serum albumin, rabbit serum albumin, thyroglobulin, keyhole limpet hemocyanin, egg ovalbumin and bovine gamma-globulins. Synthetic polyamino acids such as polylysine or polyarginine are also useful carriers. With respect to the covalent attachment of a B7 molecule protein or peptide fragments to a suitable immunogenic carrier, there are a number of chemical cross-linking agents that are known to those skilled in the art. Preferred cross-linking agents are heterobifunctional cross-linkers, which can be used to link proteins in a stepwise manner. A wide variety of heterobifunctional cross-linkers are known in the art, including succinimidyl 4-(N-maleimidomethyl)cyclohexane-1-carboxylate (SMCC), m-Maleimidobenzoyl-N-hydroxysuccinimide ester (MBS); N-succinimidyl (4-iodoacetyl)aminobenzoate (SIAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-a-methyl-a-(2-pyridyldithio)-tolune (SMPT), N-succinimidyl 3-(2-pyridyldithio) propionate (SPDP), succinimidyl 6-[3-(2-pyridyldithio)propionate]hexanoate (LC-SPDP).

In may also be desirable to simply immunize with whole cells which express a costimulatory molecule protein on their surface. Various cell lines can be used as immunogens to generate monoclonal antibodies to a B7 molecule antigen, including, but not limited to activated B cells. For example, splenic B cells can be obtained from a subject and activated with anti-immunoglobulin. Alternatively, a B cell line can be used, provided that a costimulatory molecule is expressed on the cell surface, such as the Raji cell line (B cell Burkett's lymphoma, see e.g., Freeman, G. J. et al. (1993) Science 262:909-911) or the JY B lymphoblastoid cell line (see e.g., Azuma, M. et al. (1993) Nature 366:76-79). Whole cells that can be used as immunogens to produce costimulatory molecule specific antibodies also include recombinant transfectants. For example, COS and CHO cells can be reconstituted by transfection with a costimulatory molecule cDNA, such as described by Knudson et al. (1993, PNAS 90:4003-4007); Travemor et al. (1993, Immunogenitics 37:474-477); Dougherty et al. (1991, J Exp Med 174:1-5); and Aruffo et al. (1990, Cell 61:1303-1313), to produce intact costimulatory molecule on the cell surface. These transfectant cells can then be used as immunogen to produce anti-costimulatory molecule antibodies of preselected specificity. Other examples of transfectant cells are known, particularly eukaryotic cells able to glycosylate the costimulatory molecule protein, but any procedure that works to express transfected costimulatory molecule genes on the cell surface could be used to produce the whole cell immunogen.

B. Polyclonal Anti-Costimulatory Molecule Antibodies.

Polycolonal anti-B7 antibodies can generally be raised in animals by multiple subcutaneous (sc) or intraperitoneal (ip) injections of a B7 molecule immunogen, such as the extracellular domain of a B7 molecule protein, and an adjuvant. For example, as described above, it may be useful to conjugate a B7 molecule (including fragments containing particular eptitope(s) of interest) to a protein that is immunogenic in the species to be immunized, e.g., keyhole limpet hemocyanin, serum albumin.

The route and schedule of the host animal or antibody-producing cells cultured therefrom can generally make use of established and conventional techniques for antibody stimulation and production. In an illustrative embodiment, animals are typically immunized against the immunogenic B7 molecule conjugates or derivatives by combining about 1 μg to 1 mg of conjugate with Freund's complete adjuvant and injecting the solution intradermally at multiple sites. One month later the animals are boosted with ⅕ to 1/10 the original amount of conjugate in Freund's complete adjuvant (or other suitable adjuvant) by subcutaneous injection at multiple sites. Seven to 14 days later, the animals are bled and the serum is assayed for anti-costimulatory molecule titer. Animals are boosted until the titer plateaus. Preferably, the animal is boosted with the conjugate of the same costimulatory molecule protein, but conjugated to a different protein and/or through a different cross-linking agent. Conjugates also can be made in recombinant cell culture as protein fusions. Also, aggregating agents such as alum can be used to enhance the immune response.

Such mammal-produced populations of antibody molecules are referred to as “polyclonal” because the population comprises antibodies with differing immunospecificities and affinities for a costimulatory molecule. The antibody molecules are then collected from the mammal and isolated by well known techniques such as, for example, by using DEAE Sephadex to obtain the IgG fraction. To enhance the specificity of the antibody, the antibodies may be purified by immunoaffinity chromatography using solid phase-affixed immunogen. The antibody is contacted with the solid phase-affixed immunogen for a period of time sufficient for the immunogen to immunoreact with the antibody molecules to form a solid phase-affixed immunocomplex. The bound antibodies are separated from the complex by standard techniques.

C. Monoclonal Anti-Costimulatory Molecule Antibodies. The term “monoclonal antibody” or “monoclonal antibody composition”, as used herein, refers to a population of antibody molecules that contain only one species of an antigen binding site capable of immunoreacting with a particular epitope of a B7 molecule. A monoclonal antibody composition thus typically displays a single binding affinity for a particular B7 molecule protein with which it immunoreacts. Preferably, the monoclonal antibody used in the subject method is further characterized as immunoreacting with a B7 molecule derived from humans.

Monoclonal antibodies useful in the compositions and methods of the invention are directed to an epitope of a B7 molecule antigen, such that complex formation between the antibody and the B7 molecule antigen inhibits interaction of the Bi molecule with its natural ligand(s) on the surface of immune cells, thereby inhibiting costimulation of a T cell through the B7 molecule-ligand interaction. A monoclonal antibody to an epitope of a B7 molecule can be prepared by using a technique which provides for the production of antibody molecules by continuous cell lines in culture. These include but are not limited to the hybridoma technique originally described by Kohler and Milstein (1975, Nature 256:495-497), and the more recent human B cell hybridoma technique (Kozbor et al. (1983) Immunol Today 4:72), EBV-hybridoma technique (Cole et al. (1985), Monoclonal Antibodies and Cancer Therapy, Alan R. Liss, Inc., pp. 77-96), and trioma techniques. Other methods which can effectively yield monoclonal antibodies useful in the present invention include phage display techniques (Marks et al. (1992) J Biol Chem 16007-16010).

In one embodiment, the antibody preparation applied in the subject method is a monoclonal antibody produced by a hybridoma cell line. Hybridoma fusion techniques were first introduced by Kohler and Milstein (Kohler et al. Nature (1975) 256:495-97; Brown et al. (1981) J. Immunol 127:539-46; Brown et al. (1980) J Biol Chem 255:4980-83; Yeh et al. (1976) PNAS 76:2927-31; and Yeh et al. (1982) Int. J. Cancer 29:269-75). Thus, the monoclonal antibody compositions of the present invention can be produced by the following method, which comprises the steps of:

(a) Immunizing an animal with a B7 molecule. The immunization is typically accomplished by administering a B7 molecule immunogen to an immunologically competent mammal in an immunologically effective amount, i.e., an amount sufficient to produce an immune response. Preferably, the mammal is a rodent such as a rabbit, rat or mouse. The mammal is then maintained for a time period sufficient for the mammal to produce cells secreting antibody molecules that immunoreact with the B7 molecule immunogen. Such immunoreaction is detected by screening the antibody molecules so produced for immunoreactivity with a preparation of the immunogen protein. Optionally, it may be desired to screen the antibody molecules with a preparation of the protein in the form in which it is to be detected by the antibody molecules in an assay, e.g., a membrane-associated form of a B7 molecule. These screening methods are well known to those of skill in the art.

(b) A suspension of antibody-producing cells removed from each immunized mammal secreting the desired antibody is then prepared. After a sufficient time, the mouse is sacrificed and somatic antibody-producing lymphocytes are obtained. Antibody-producing cells may be derived from the lymph nodes, spleens and peripheral blood of primed animals. Spleen cells are preferred, and can be mechanically separated into individual cells in a physiologically tolerable medium using methods well known in the art. Mouse lymphocytes give a higher percentage of stable fusions with the mouse myelomas described below. Rat, rabbit and frog somatic cells can also be used. The spleen cell chromosomes encoding desired immunoglobulins are immortalized by fusing the spleen cells with myeloma cells, generally in the presence of a fusing agent such as polyethylene glycol (PEG). Any of a number of myeloma cell lines may be used as a fusion partner according to standard techniques; for example, the P3-NS1/1-Ag4-1, P3-x63-Ag8.653 or Sp2/O-Ag14 myeloma lines. These myeloma lines are available from the American Type Culture Collection (ATCC), Rockville, Md.

The resulting cells, which include the desired hybridomas, are then grown in a selective medium, such as HAT medium, in which unfused parental myeloma or lymphocyte cells eventually die. Only the hybridoma cells survive and can be grown under limiting dilution conditions to obtain isolated clones. The supernatants of the hybridomas are screened for the presence of antibody of the desired specificity, e.g., by immunoassay techniques using the antigen that has been used for immunization. Positive clones can then be subcloned under limiting dilution conditions and the monoclonal antibody produced can be isolated. Various conventional methods exist for isolation and purification of the monoclonal antibodies so as to free them from other proteins and other contaminants. Commonly used methods for purifying monoclonal antibodies include ammonium sulfate precipitation, ion exchange chromatography, and affinity chromatography (see, e.g., Zola et al. in Monoclonal Hybridoma Antibodies: Techniques And Applications, Hurell (ed.) pp. 51-52 (CRC Press 1982)). Hybridomas produced according to these methods can be propagated in vitro or in vivo (in ascites fluid) using techniques known in the art.

Generally, the individual cell line may be propagated in vitro, for example in laboratory culture vessels, and the culture medium containing high concentrations of a single specific monoclonal antibody can be harvested by decantation, filtration or centrifugation. Alternatively, the yield of monoclonal antibody can be enhanced by injecting a sample of the hybridoma into a histocompatible animal of the type used to provide the somatic and myeloma cells for the original fusion. Tumors secreting the specific monoclonal antibody produced by the fused cell hybrid develop in the injected animal. The body fluids of the animal, such as ascites fluid or serum, provide monoclonal antibodies in high concentrations. When human hybridomas or EBV-hybridomas are used, it is necessary to avoid rejection of the xenograft injected into animals such as mice. Immunodeficient or nude mice may be used or the hybridoma may be passaged first into irradiated nude mice as a solid subcutaneous tumor, cultured in vitro and then injected intraperitoneally into pristane primed, irradiated nude mice which develop ascites tumors secreting large amounts of specific human monoclonal antibodies.

Media and animals useful for the preparation of these compositions are both well known in the art and commercially available and include synthetic culture media, inbred mice and the like. An exemplary synthetic medium is Dulbecco's minimal essential medium (DMEM; Dulbecco et al. (1959) Virol 8:396) supplemented with 4.5 gm/l glucose, 20 mM glutamine, and 20% fetal caf serum. An exemplary inbred mouse strain is the Balb/c.

D. Humanized or Chimeric Anti-B7 Molecule Antibodies. When antibodies produced in non-human subjects are used therapeutically in humans, they are recognized to varying degrees as foreign and an immune response may be generated in the patient. One approach for minimizing or eliminating this problem, which is preferable to general immunosuppression, is to produce chimeric antibody derivatives, i.e., antibody molecules that combine a non-human animal variable region and a human constant region. Such antibodies are the equivalents of the monoclonal and polyclonal antibodies described above, but may be less immunogenic when administered to humans, and therefore more likely to be tolerated by the patient.

Chimeric mouse-human monoclonal antibodies (i.e., chimeric antibodies) reactive with a costimulatory molecule can be produced, for example, by techniques recently developed for the production of chimeric antibodies. Methods of humanizing antibodies are known in the art. The humanized antibody may be the product of an animal having transgenic human immunoglobulin constant region genes (see for example International Patent Applications WO 90/10077 and WO 90/04036). Alternatively, the antibody of interest may be engineered by recombinant DNA techniques to substitute the CH1, CH2, CH3, hinge domains, and/or the framework domain with the corresponding human sequence (see WO 92/02190).

The use of Ig cDNA for construction of chimeric immunoglobulin genes is known in the art (Liu et al. (1987) P.N.A.S. 84:3439 and (1987) J. Immunol. 139:3521). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods.

Additionally, recombinant anti-B7 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira, et al. European Patent Application 184,187; Taniguchi, M., European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060. In addition, humanized antibodies can be made according to standard protocols such as those disclosed in U.S. Pat. Nos. 5,777,085; 5,530,101; 5,693,762; 5,693,761; 5,882,644; 5,834,597; 5,932,448; or 5,565,332.

For example, an antibody may be humanized by grafting the desired CDRs onto a human framework, e.g., according to EP-A-0239400. A DNA sequence encoding the desired reshaped antibody can therefore be made beginning with the human DNA whose CDRs it is wished to reshape. The rodent variable domain amino acid sequence containing the desired CDRs is compared to that of the chosen human antibody variable domain sequence. The residues in the human variable domain are marked that need to be changed to the corresponding residue in the rodent to make the human variable region incorporate the rodent CDRs. There may also be residues that need substituting, e.g., adding to or deleting from the human sequence. Oligonucleotides can be synthesized that can be used to mutagenize the human variable domain framework to contain the desired residues. Those oligonucleotides can be of any convenient size.

Alternatively, humanization may be achieved using the recombinant polymerase chain reaction (PCR) methodology of WO 92/07075. Using this methodology, a CDR may be spliced between the framework regions of a human antibody. In general, the technique of WO 92/07075 can be performed using a template comprising two human framework regions, AB and CD, and between them, the CDR which is to be replaced by a donor CDR. Primers A and B are used to amplify the framework region AB, and primers C and D used to amplify the framework region CD. However, the primers B and C each also contain, at their 5′ ends, an additional sequence corresponding to all or at least part of the donor CDR sequence. Primers B and C overlap by a length sufficient to permit annealing of their 5′ ends to each other under conditions which allow a PCR to be performed. Thus, the amplified regions AB and CD may undergo gene splicing by overlap extension to produce the humanized product in a single reaction.

In one method, humanized anti-B7 antibodies can be made by joining polynucleotides encoding portions of immunoglobulins capable of binding B7 to polynucleotides encoding appropriate human framework regions. Exemplary humanization methods can be found, e.g., in Queen et al. Proc. Natl. Acad. Sci. 1989. 86:10029 or U.S. Pat. Nos. 5,585,089 or 5,693,762 the teachings of which are incorporated herein in their entirety.

In another embodiment, antibody chains or specific binding pair members can be produced by recombination between vectors comprising nucleic acid molecules encoding a fusion of a polypeptide chain of an antibody and a component of a replicable genetic display package and vectors containing nucleic acid molecules encoding a second polypeptide chain of a single binding pair member using techniques known in the art, e.g., as described in U.S. Pat. Nos. 5,565,332, 5,871,907, or 5,733,743.

E. Combinatorial Anti-Costimulatory Molecule Antibodies. Both monoclonal and polyclonal antibody compositions of the invention can also be produced by other methods well known to those skilled in the art of recombinant DNA technology. An alternative method, referred to as the “combinatorial antibody display” method, has been developed to identify and isolate antibody fragments having a particular antigen specificity, and can be utilized to produce monoclonal anti-costimulatory molecule antibodies, as well as a polyclonal anti-costimulatory molecule population (Sastry et al. (1989) PNAS 86:5728; Huse et al. (1989) Science 246:1275; and Orlandi et al. (1989) PNAS 86:3833). After immunizing an animal with a costimulatory molecule immunogen as described above, the antibody repertoire of the resulting B-cell pool is cloned. Methods are generally known for directly obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies (Larrick et al. (1991) Biotechniques 11: 152-156). A similar strategy can also been used to amplify human heavy and light chain variable regions from human antibodies (Larrick et al. (1991) Methods. Companion to Methods in Enzymology 2:106-110). The ability to clone human immunoglobulin V-genes takes on special significance in light of advancements in creating human antibody repertoires in transgenic animals (see, for example, Bruggeman et al. (1993) Year Immunol 7:33-40; Tuaillon et al. (1993) PNAS 90:3720-3724; Bruggeman et al. (1991) Eur J Immunol 21:1323-1326; and Wood et al. PCT publication WO 91/00906).

In an illustrative embodiment, RNA is isolated from activated B cells of, for example, peripheral blood cells, bone marrow, or spleen preparations, using standard protocols (e.g., U.S. Pat. No. 4,683,202; Orlandi, et al. PNAS (1989) 86:3833-3837; Sastry et al., PNAS (1989) 86:5728-5732; and Huse et al. (1989) Science 246:1275-1281.) First-strand cDNA is synthesized using primers specific for the constant region of the heavy chain(s) and each of the κ and λ light chains, as well as primers for the signal sequence. Using variable region PCR primers, the variable regions of both heavy and light chains are amplified, each alone or in combinantion, and ligated into appropriate vectors for further manipulation in generating the display packages. Oligonucleotide primers useful in amplification protocols may be unique or degenerate or incorporate inosine at degenerate positions. Restriction endonuclease recognition sequences may also be incorporated into the primers to allow for the cloning of the amplified fragment into a vector in a predetermined reading frame for expression.

The V-gene library cloned from the inmunization-derived antibody repertoire can be expressed by a population of display packages, preferably derived from filamentous phage, to form an antibody display library. Ideally, the display package comprises a system that allows the sampling of very large variegated antibody display. libraries, rapid sorting after each affinity separation round, and easy isolation of the antibody gene from purified display packages. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating a variegated anti-costimulatory molecule antibody display library can be found in, for example, the Ladner et al. U.S. Pat. No. 5,223,409; the Kang et al. International Publication No. WO 92/18619; the Dower et al. International Publication No. WO 91/17271; the Winter et al. International Publication WO 92/20791; the Markland et al. International Publication No. WO 92/15679; the Breitling et al. International Publication WO 93/01288; the McCafferty et al. International Publication No. WO 92/01047; the Garrard et al. International Publication No. WO 92/09690; the Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

In certain embodiments, the V region domains of heavy and light chains can be expressed on the same polypeptide, joined by a flexible linker to form a single-chain Fv fragment, and the scFV gene subsequently cloned into the desired expression vector or phage genome. As generally described in McCafferty et al., Nature (1990) 348:552-554, complete V_{H} and V_{L} domains of an antibody, joined by a flexible (Gly₄-Ser)₃ linker can be used to produce a single chain antibody which can render the display package separable based on antigen affinity. Isolated scFV antibodies immunoreactive with a costimulatory molecule can subsequently be formulated into a pharmaceutical preparation for use in the subject method.

F. Hybridomas and Methods of Preparation. Hybridomas useful in the present invention are those characterized as having the capacity to produce a monoclonal antibody which will specifically immunoreact with a costimulatory molecule. As described below, the hybridoma cell producing anti-costimulatory molecule antibody can be directly implanted into the recipient animal in order to provide a constant source of antibody. The use of immuno-isolatory devices to encapsulate the hybridoma culture can prevent immunogenic response against the implanted cells, as well as prevent unchecked proliferation of the hybridoma cell in an immunocompromised host. A preferred hybridoma of the present invention is characterized as producing antibody molecules that specifically immunoreact with a costimulatory molecule expressed on the cell surfaces of activated human B cells.

Methods for generating hybridomas that produce, e.g., secrete, antibody molecules having a desired immunospecificity, i.e., having the ability to bind to a particular costimulatory molecule, and/or an identifiable epitope of a costimulatory molecule, are well known in the art. Particularly applicable is the hybridoma technology described by Niman et al. (1983) PNAS 80:4949-4953; and by Galfre et al. (1981) Meth. Enzymol. 73:3-46.

In another exemplary method, transgenic mice carrying human antibody repertoires can be immunized with a human costimultory molecule. Splenocytes from these immunized transgenic mice can then be used to create hybridomas that secrete human monoclonal antibodies specifically reactive with a human costimultory molecule (see, e.g., Wood et al. PCT publication WO 91/00906, Kucherlapati et al. PCT publication WO 91/10741; Lonberg et al. PCT publication WO 92/03918; Kay et al. PCT publication 92/03917; Lonberg, N. et al. (1994) Nature 368:856-859; Green, L. L. et al. (1994) Nature Genet. 7:13-21; Morrison, S. L. et al. (1994) Proc. Natl. Acad. Sci. USA 81:6851-6855; Bruggeman et al. (1993) Year Immunol 7:33-40; Tuaillon et al. (1993) PNAS 90:3720-3724; and Bruggeman et al. (1991) Eur J Immunol 21:1323-1326).

The term “antibody” as used herein is intended to include fragments thereof which are also specifically reactive with a costimulatory molecule as described herein. Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments.

Antibodies made using these or other methods can be tested to determine whether they inhibit a costimulatory signal in a T cell using the methods described below.

In one embodiment an antibody for use in the claimed methods binds to both B7-1 and B7-2. In making such an antibody, for example, portions of the extracellular domain which are conserved between the two costimulatory molecules can be used as the immunogen. See, e.g., Metzler et al. 1997 Nat Struct. Biol. 4:527).

In one embodiment, an antibody for use in the claimed methods is an antibody which binds to B7-1. Such antibodies are known in the art-or can be made as set forth above using a B7-1 molecule or a portion thereof as an immunogen and screened using the methods set forth above or other standard methods. Examples of B7-1 antibodies include those taught in U.S. Pat. No. 5,747,034 and in McHugh et al. 1998. Clin. Immunol. Immunopathol. 87:50 or Rugtveit et al. 1997. Clin Exp. Immunol. 110: 104.

In another embodiment, an antibody for use in the claimed methods is an antibody which binds to B7-2. Such antibodies are known in the art or can be made as set forth above using a B7-2 molecule or a portion thereof as an immunogen and screened using the methods set forth above or other standard methods. Examples of B7-2 antibodies include those taught in Rugtveit et al. 1997. Clin Exp. Immunol. 110:104.

In one embodiment, the claimed methods employ a combination of an antibody which binds to B7-1 and an antibody which binds to B7-2.

III. Expression of Antibodies

An antibody, or antigen binding portion, of the invention can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To express an antibody having anti-B7 activity, DNA fragments encoding the light and heavy chain variable regions are first obtained. These DNAs can be obtained by amplification and modification of germline light and heavy chain variable sequences using the polymerase chain reaction (PCR). Germline DNA sequences for human heavy and light chain variable region genes are known in the art (see e.g., the “Vbase” human germline sequence database; see also Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{κ} Segments Reveals a Strong Bias in their Usage” Eur. J. Immunol. 24:827-836; the contents of each of which are expressly incorporated herein by reference).

To express the antibodies, or antigen binding portions of the invention, DNAs encoding partial or full-length light and heavy chains, obtained as described above, can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the antibody-related light or heavy chain sequences, the expression vector may already carry antibody constant region sequences. For example, one approach to converting the antibody-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

The nucleic acid sequences of the present invention capable of ultimately expressing the desired antibodies can be formed from a variety of different polynucleotides (genomic or cDNA, RNA, synthetic oligonucleotides, etc.) and components (e.g., V, J, D, and C regions), as well as by a variety of different techniques. Joining appropriate genomic and synthetic sequences is presently the most common method of production, but CDNA sequences may also be utilized (see, European Patent Publication No. 0239400 and Reichmann, L. et al., Nature 332, 323-327 (1988), both of which are incorporated herein by reference).

In addition to the antibody chain genes, the recombinant expression vectors of the invention carry regulatory sequences that control the expression of the antibody chain genes in a host cell. The term “regulatory sequence” includes promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It will be understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to a B7 molecule. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than a B7 molecule by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

Antibodies, (e.g., whole antibodies, their dimers, individual light and heavy chains, or other immunoglobulin forms of the present invention), can be purified according to standard procedures of the art, including ammonium sulfate precipitation, affinity columns, column chromatography, gel electrophoresis and the like (see, generally, R. Scopes, “Protein Purification”, Springer-Verlag, N.Y. (1982)). Substantially pure immunoglobulins of at least about 90 to 95% homogeneity are preferred, and 98 to 99% or more homogeneity most preferred, for pharmaceutical uses. Once purified, partially or to homogeneity as desired, the polypeptides may then be used therapeutically (including extracorporeally) or in developing and performing assay procedures, immunofluorescent stainings, and the like. (See, generally, Immunological Methods, Vols. I and II, Lefkovits and Pernis, eds., Academic Press, New York, N.Y. (1979 and 1981)).

In view of the foregoing, another aspect of the invention pertains to nucleic acid, vector and host cell compositions that can be used for recombinant expression of the antibodies and antibody portions of the invention.

It will be appreciated by the skilled artisan that nucleotide sequences encoding antibodies, or portions thereof (e.g., a CDR domain, such as a CDR3 domain), can be derived from the nucleotide sequences encoding the antibody using the genetic code and standard molecular biology techniques.

The invention also provides recombinant expression vectors encoding both an antibody heavy chain and an antibody light chain. For example, in one embodiment, the invention provides a recombinant expression vector encoding:

a) an antibody light chain having a variable region of an anti-B7 antibody or a humanized form thereof; and

b) an antibody heavy chain having a variable region of an anti-B7 antibody or a humanized form thereof.

The invention also provides host cells into which one or more of the recombinant expression vectors of the invention have been introduced. Preferably, the host cell is a mammalian host cell, more preferably the host cell is a CHO cell, an NS0 cell or a COS cell.

Still further the invention provides a method of synthesizing an antibody of the invention by culturing a host cell of the invention in a suitable culture medium until an antibody of the invention is synthesized. The method can further comprise isolating the antibody from the culture medium.

In addition, several B7 antibodies are known or are readily available (see, e.g., U.S. Pat. No. 5,869,050; Powers G. D., et al. (1994) Cell. Immunol. 153, 298-311; Freedman, A. S. et al. (1987) J. Immunol. 137:3260-3267; Freeman, G. J. et al. (1989) J. Immunol. 143:2714-2722; Freeman, G. L. et al. (1991) J. Exp. Med. 174:625-631; Freeman, G. J. (1993) Science 262:909-911; Wo 96/40915). Such antibodies are also commercially available, e.g. from R&D Systems (Minneapolis, Minn.) and Research Diagnostics (Flanders, N.J.).

IV Therapeutic Uses of Anti-B7 Antibodies and Rapamycin Compounds in Inhibition of Immune Responses

The antibodies of the current invention can be used therapeutically to inhibit immune responses through blocking receptor:ligand interactions necessary for costimulation of the T cell. Antibodies for use in the instant invention can be identified by their ability to inhibit T cell proliferation and/or cytokine production when added to an in vitro costimulation assay as described herein. The ability of blocking antibodies to inhibit T cell functions may result in immunosuppression and/or tolerance when these antibodies are administered in vivo.

Assays to test the blocking activity of anti-B7 antibodies for use in therapeutic applications take advantage of the functional characteristics of the B7 antigen. As previously set forth, the ability of T cells to synthesize cytokines depends not only on occupancy or cross-linking of the T cell receptor for antigen (“the primary activation signal provided by, for example anti-CD3, or phorbol ester to produce an “activated T cell”), but also on the induction of a costimulatory signal, in this case, by interaction with a B7 molecule. The binding of B7 to its natural ligand(s) on, for example, CD28⁺ T cells, has the effect of transmitting a signal to the T cell that induces the production of increased levels of cytokines, particularly of interleukin-2, which in turn stimulates the proliferation of the T lymphocytes. Other assays for B7 function thus involve assaying for the synthesis of cytokines, such as interleukin-2, interleukin-4 or other known or unknown novel cytokines, and/or assaying for T cell proliferation by CD28⁺ T cells which have received a primary activation signal.

The ability of an anti-B7 antibody to inhibit (or completely block the normal B7 costimulatory signal and induce a state of anergy) can be determined using subsequent attempts at stimulation of T cells with antigen presenting cells that express cell surface B7 and present antigen. If the T cells are unresponsive to the subsequent activation attempts, as determined by IL-2 synthesis and T cell proliferation, a state of anergy has been induced. See, e.g., Gimmi, C. D. et al. (1993) Proc. Natl. Acad. Sci. USA 90, 6586-6590; and Schwartz (1990) Science, 248, 1349-1356, for assay systems that can used as the basis for an assay in accordance with the present invention. The ability of an anti-B7 antibody to block or inhibit T cell costimulation is assayed by adding an anti-B7 antibody to be tested and a primary activation signal such as antigen in association with Class II MHC to a T cell culture and assaying the culture supernatant for interleukin-2, gamma interferon, or other known or unknown cytokine. For example, any one of several conventional assays for interleukin-2 can be employed, such as the assay described in Proc. Natl. Acad. Sci. USA, 86:1333 (1989) which is incorporated herein by reference. A kit for an assay for the production of interferon is also available from Genzyme Corporation (Cambridge, Mass.). T cell proliferation can also be measured by a assaying [³H] thymidine incorporation.

The methods of the current invention can be used therapeutically to inhibit immune responses in a subject that would benefit from such a reduction in immune response. Downregulation of an immune response may be in the form of inhibiting or blocking an immune response already in progress or may involve preventing the induction of an immune response. The functions of activated T cells may be inhibited by suppressing immune cell responses or by inducing specific tolerance, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent. Exemplary situations in which the subject combination therapies can be employed are set forth below.

A. Organ Transplantation/GVHD: Anti-B7-1 and/or B7-2 antibody in combination with Rapamycin is useful e.g., in situations of tissue, skin and organ transplantation and in graft-versus-host disease (GVHD). For example, blockage of immune cell function should result in reduced tissue destruction in tissue transplantation. Typically, in tissue transplants, rejection of the transplant is initiated through its recognition as foreign by T cells, followed by an immune reaction that destroys the transplant. The administration of Anti-B7-1 and/or B7-2 antibody in combination with Rapamycin, prior to and/or post transplantation may block or inhibit interaction of the B7 molecules with their natural ligand(s) on immune cells. Blocking B7 function in this manner prevents cytokine synthesis by immune cells, such as T cells, and thus acts as an immunosuppressant. Moreover, the lack of costimulation may also be sufficient to anergize the T cells, thereby inducing tolerance in a subject.

The efficacy of particular combination therapy in preventing organ transplant rejection or GVHD can be assessed using animal models that are predictive of efficacy in humans.

After prolonged graft acceptance, an antibody-treated animal can be sacrificed and the lymphocytes from the “tolerant” animal can be tested for their functional responses. These responses can be compared with those of lymphocytes from a control (non-transplanted) animal, and results are normalized as a percentage of the control response. The T cell proliferative response to polyclonal activators or third party stimulator cells can be examined. Additionally, the thymus and spleen from the untreated and treated animals can be compared in size, cell number and cell type (e.g. by flow cytometic analyses of thymus, lymph nodes and spleen cells). Specific tolerance in the treated animals is indicated by the ability of the T cells to respond to mitogens and third party stimulators but not to the cells used in the transplant.

B. Autoimmune Diseases: Blocking B7 function, by use of anti-B7-1 and/or B7-2 antibody in combination with Rapamycin is also useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of at least one anti-B7 antibody and Rapamycin can be used to inhibit T cell activation and prevent production of autoantibodies or T cell-derived cytokines which may be involved in the disease process. Additionally, the claimed combination therapies may induce antigen-specific tolerance of autoreactive T cells which could lead to long-term relief from the disease.

The subject therapies can be used to treat a variety of immune diseases, e.g., Examples of autoimmune diseases or disorders associated with an inappropriate or abnormal immune response include rheumatoid arthritis, juvenile rheumatoid arthritis, psoriatic arthritis, allergies, contact dermatitis, psoriasis, leprosy reversal reactions, erythema nodosum leprosum, autoimmune uveitis, multiple sclerosis, allergic encephalomyelitis, systemic lupus erythematosus, acute necrotizing hemorrhagic encephalopathy, idiopathic bilateral progressive sensorineural hearing loss, aplastic anemia, pure red cell anemia, idiopathic thrombocytopenia, polychondritis, scleroderma, Wegener's granulomatosis, chronic active hepatitis, myasthenia gravis, Stevens-Johnson syndrome, idiopathic sprue, lichen planus, Crohn's disease, Graves ophthalmopathy, sarcoidosis, primary biliary cirrhosis, primary juvenile diabetes, dry eye associated with Sjogren's syndrome, uveitis posterior, and interstitial lung fibrosis.

The efficacy of blocking reagents in preventing or alleviating autoimmune disorders can be determined using a number of well-characterized animal models of human autoimmune diseases. Examples include murine experimental autoimmune encephalitis, systemic lupus erythmatosis in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

C. Allergy: The IgE antibody response in atopic allergy is highly T cell dependent and, thus, anti-B7-1 and/or B7-2 antibody in combination with Rapamycin may be useful therapeutically in the treatment of allergy and allergic reactions. The subject combination therapies can be administered to an allergic subject to inhibit T cell mediated allergic responses in the subject. Inhibition of costimulation of T cells may be accompanied by exposure to allergen in conjunction with appropriate MHC molecules. Allergic reactions may be systemic or local in nature, depending on the route of entry of the allergen and the pattern of deposition of IgE on mast cells or basophils. Thus, it may be necessary to inhibit T cell mediated allergic responses locally or systemically by proper administration of anti-B lymphocyte antigen antibodies.

Other exemplary disorders that the instant methods can be used to treat include: inhibition of transplantation of organs or tissue, graft-versus-host diseases brought about by medulla ossium transplantation; rheumatoid arthritis, systemic lupus erythematosus, Hashimoto's thyroiditis, multiple sclerosis, myasthenia gravis, type I diabetes uveitis, juvenile-onset or recent-onset diabetes mellitus, posterior uveitis, allergic encephalomyelitis, glomerulonephritis, inflammatory and hyperproliferative skin diseases, psoriasis, atopical dermatitis, contact dermatitis, eczematous dermatitises, seborrhoeis dermatitis, urticaria, Lupus erythematosus, acne, Alopecia areata, keratoconjunctivitis, vernal conjunctivitis, pollen allergies, bronchial asthma, allergic asthma, intrinsic asthma, extrinsic asthma and dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis,

V Administration of Additional Agents

In one embodiment, anti-B7-1 and/or B7-2 antibody in combination with Rapamycin can be used with other immunosuppressive agents, e.g., antibodies against other immune cell surface markers (e.g., CD40) or against cytokines, other fusion proteins, e.g., CTLA4Ig, or other immunosuppressive drugs (e.g., cyclophosphamide, cyclosporin A, FK506, or steroids). In a preferred embodiment of the invention the subject method comprises administering a combination of two anti-B7 antibodies and a Rapamycin compound to a subject with systemic lupus erythematosis.

As used herein the term “Rapamycin compound” includes the neutral tricyclic compound Rapamycin, Rapamycin derivatives, Rapamycin analogs, and other macrolide compounds which are thought to have the same mechanism of action as Rapamycin (e.g., inhibition of cytokine function). The language “Rapamycin compounds” includes compounds with structural similarity to Rapamycin, e.g., compounds with a similar macrocyclic structure, which have modified to enhance therapeutic benefit. Exemplary Rapamycin compounds suitable for use in the invention are known in the art. In addition, other methods in which Rapamycin has been administered are known in the art. For example, see WO 95/22972, WO 95/16691, WO 95/04738, U.S. Pat. Nos. 6,015,809; 5,989,591; U.S. Pat. Nos. 5,567,709; 5,559,112; 5,530,006; 5,484,790; 5,385,908; 5,202,332; 5,162,333; 5,780,462; 5,120,727.

The language “FK506-like compounds” includes FK506, and FK506 derivatives and analogs, e.g., compounds with structural similarity to FK506, e.g., compounds with a similar macrocyclic structure, which have modified to enhance therapeutic benefit. Examples of FK506 like compounds include, for example, those described in WO 00/01385. Preferably, the language “Rapamycin compound” as used herein does not include FK506-like compounds.

VI. Administration of Therapeutic Compositions

The antibodies of the invention are administered to subjects in a biologically compatible form suitable for pharmaceutical administration in vivo to inhibit immune responses. By “biologically compatible form suitable for administration in vivo” is meant a form of the protein to be administered in which any toxic effects are outweighed by the therapeutic effects of the antibody. The term subject is intended to include living organisms in which an immune response can be elicited, e.g., mammals. Examples of subjects include humans, dogs, cats, mice, rats, and transgenic species thereof. Administration of an antibody of the invention as described herein can be in any pharmacological form including a therapeutically active amount of anti-B7 antibody alone or in combination with an antibody reactive with another B lymphocyte antigen (e.g., B7-1) and a pharmaceutically acceptable carrier. Administration of a therapeutically active amount of the therapeutic compositions of the present invention is defined as an amount effective, at dosages and for periods of time necessay to achieve the desired result. For example, a therapeutically active amount of an anti-B7 antibody may vary according to factors such as the disease state, age, sex, and weight of the individual, and the ability of peptide to elicit a desired response in the individual. A dosage regime may be adjusted to provide the optimum therapeutic response. For example, several divided doses may be administered daily or the dose may be proportionally reduced as indicated by the exigencies of the therapeutic situation.

The active compound (e.g., antibody) may be administered in a convenient manner such as by injection (subcutaneous, intravenous, etc.), oral administration, inhalation, transdermal application, or rectal administration. Depending on the route of administration, the active compound may be coated in a material to protect the compound from the action of enzymes, acids and other natural conditions which may inactivate the compound.

To administer an anti-B7 antibody by other than parenteral administration, it may be necessary to coat the peptide with, or co-administer the antibody with, a material to prevent its inactivation. An anti-B7 antibody may be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Exemplary adjuvants include alum, resorcinols, non-ionic surfactants such as polyoxyethylene oleyl ether and n-hexadecyl polyethylene ether. Enzyme inhibitors include pancreatic trypsin inhibitor, diisopropylfluorophosphate (DEP) and trasylol. Liposomes include water-in-oil-in-water emulsions as well as conventional liposomes (Strejan et al., (1984) J. Neuroimmunol 7:27).

The active compound may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition will preferably be sterile and fluid to the extent that easy syringability exists. It will preferably be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifingal agents, for example, parabens, chlorobutanol, phenol, asorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating active compound (e.g., anti-B7 antibody and/or Rapamycin) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (e.g., antibody) plus any additional desired ingredient from a previously sterile-filtered solution thereof.

When the active compound is suitably protected, as described above, the protein may be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In one embodiment of the present invention a therapeutically effective amount of an antibody to a protein is administered to a subject. As defined herein, a therapeutically effective amount of antibody (i.e., an effective dosage) ranges from about 0.001 to 50 mg/kg body weight, preferably about 0.01 to 40 mg/kg body weight, more preferably about 0.1 to 30 mg/kg body weight, about 1 to 25 mg/kg, 2 to 20 mg/kg, 5 to 15 mg/kg, or 7 to 10 mg/kg body weight. The optimal dose of the antibody given may even vary in the same patient depending upon the time at which it is administered.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of assays designed to monitor transplant status (e.g., whether rejection or an immune response in the subject has occurred) as known in the art or as described herein.

In one embodiment, a pharmaceutical composition for injection could be made up to contain 1 ml sterile buffered water, and 1 to 50 mg of antibody. A typical composition for intravenous infusion could be made up to contain 250 ml of sterile Ringer's solution, and 150 mg of antibody. Actual methods for preparing parenterally administrable compositions will be known or apparent to those skilled in the art and are described in more detail in, for example, Remington's Pharmaceutical Science, 15th ed., Mack Publishing Company, Easton, Pa. (1980), which is incorporated herein by reference. The compositions comprising the present antibodies can be administered for prophylactic and/or therapeutic treatments. In therapeutic application, compositions can be administered to a patient already suffering from a disease, in an amount sufficient to cure or at least partially arrest the disease and its complications. An amount adequate to accomplish this is defined as a “therapeutically effective dose.” Amounts effective for this use will depend upon the clinical situation and the general state of the patient's own immune system. For example, doses for preventing transplant rejection may be lower than those given if the patient presents with clinical symptoms of rejection. Single or multiple administrations of the compositions can be carried out with dose levels and pattern being selected by the treating physician. In any event, the pharmaceutical formulations should provide a quantity of the antibody(ies) of this invention sufficient to effectively treat the patient.

Dose administration can be repeated depending upon the pharmacokinetic parameters of the dosage formulation and the route of administration used. It is also provided that certain protocols may allow for one or more agents describe herein to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, olyvinylpyrrolidone, cellulose, gelatin, syrup, methyl cellulose, methyl- and propylhydroxybenzoates, talc. magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and/or substances which promote absorption such as, for example, surface active agents.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier.

The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method for the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Thus, the dosage of any of the subject agents, e.g., antibodies or immunosuppressive drug can be easily determined by one of ordinary skill in the art. The dose may vary depending on the age, health and weight of the recipient, the extent of disease, kind of concurrent treatment, if any, frequency of treatment and the nature of the effect desired. Exemplary doses for the anti-B7 antibodies of the invention include 3 mg/kg, 5, mg/kg, 10 mg/kg, 15 mg/kg, or 20 mg/kg. It should be noted that the dose of antibody given to one subject may vary during the course of the treatment.

The pharmaceutical compositions can be included in a container, pack, or dispenser together with instructions for administration. Kits for practice of the instant invention are also provided. For example, such a diagnostic kit comprises an antibody conjugated to a toxin. The kit can further comprise a means for administering the antibody conjugate, e.g., one or more syringes. The kit can come packaged with instructions for use.

The contents of all references, pending patent applications and published patents, cited throughout this application are hereby expressly incorporated by reference. Each reference disclosed herein is incorporated by reference herein in its entirety. Any patent application to which this application claims priority is also incorporated by reference herein in its entirety.

### EXAMPLES

### Example 1

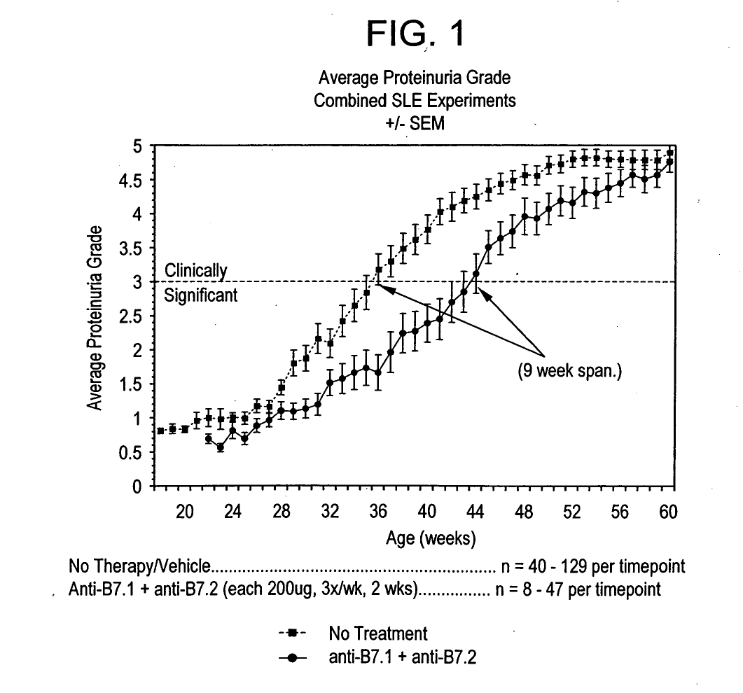

The degree of urine Albumin is indicative of disease progression since a healthy kidney prevents the protein from entering the urine. Autoimmune diseases such as SLE create immune complexes which damage the filtering system of the kidneys, allowing Albumin to be excreted. Generally, the more ill the animal, the higher the proteinuria grade. Preliminary experiments confirmed the disease progression in untreated NZB/NZW F1 female mice and provided evidence of the therapeutic benefit of costimulatory blockade with anti-B7 antibodies (FIG. 1). Short-term combination anti-B7-1 and anti-B7-2 monoclonal antibody therapy during the onset of clinical disease appeared to slow disease progression for a 2-month period by transiently ameliorating morbidity, decreasing auto-antibody production, decreasing the proteinuria grade and by improving long-term survival in lupus prone mice (FIG. 1 and observed data).

### Example 2

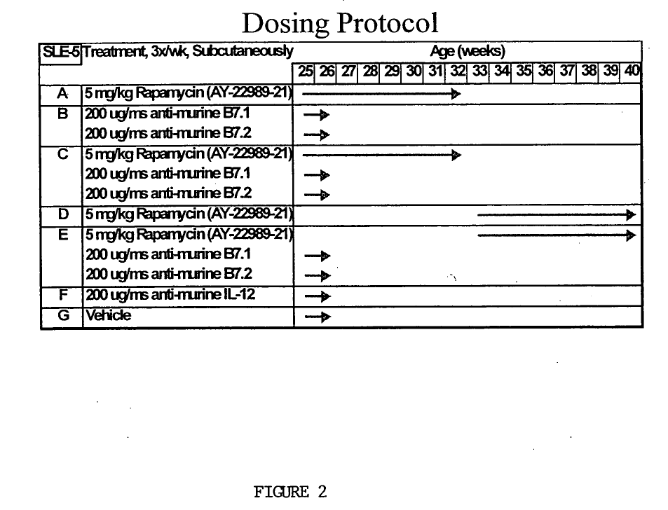

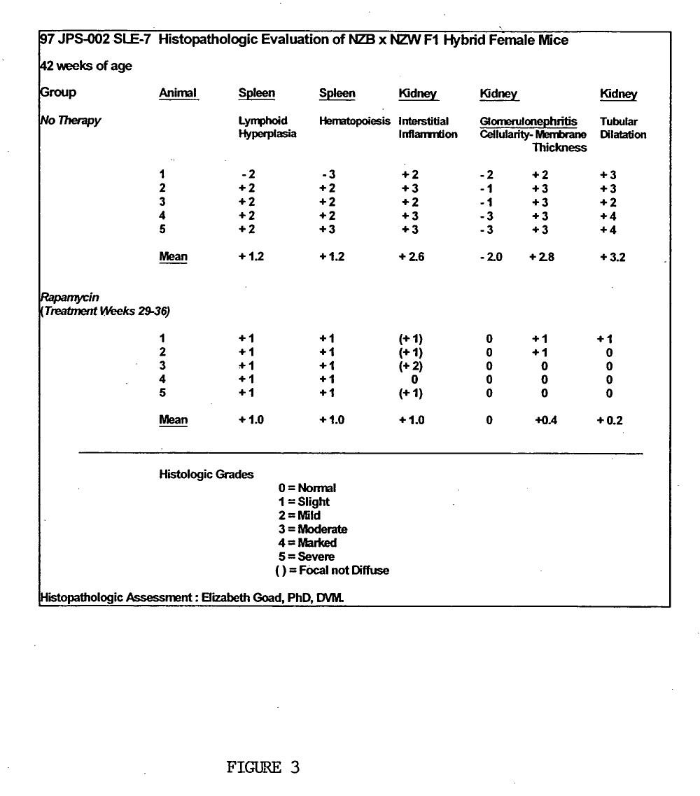

NZB/NZW F1 (B/W) mice develop autoimmune disease that resembles human systemic erythematosus (SLE). Mice, aged at least 5 months (˜25 weeks) and experiencing onset of renal disease, were chosen for these studies to more closely resemble the clinical presentation of patients with established SLE. Mice were treated according to the dosing protocol provided in FIG. 2. “Early Rapamycin Dosing” consisted of beginning treatment at week 25. “Late Rapamycin Dosing” consisted of beginning treatment at week 33. Histopathologic Evaluation of treated and untreated NZB/NZW F1 (B/W) mice were performed following autopsy and H&E staining of kidney tissue sections examined at 100× magnification (FIG. 3).

### Example 3

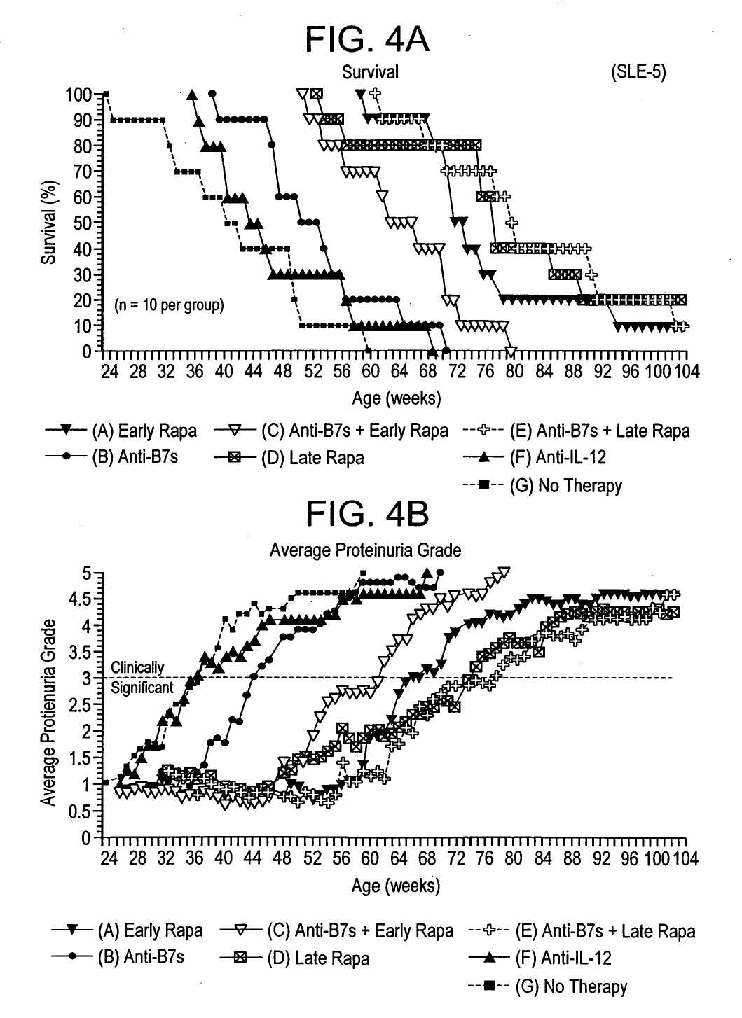

The effect of brief therapy with anti-murine B7.1 and anti-B7.2 monoclonal antibodies was compared with combination therapy with the immunosuppressant Rapamycin. Cellular and humoral immune responses were monitored in addition to renal histological changes. With a 2 week course of the anti-B7-antibodies, or when therapy was combined with an 8 week course of Rapamycin treatment, survival was prolonged (FIG. 4A). This observation was concomitant with reduced proteinuria, as opposed to increased proteinuria and mortality seen in untreated mice (FIG. 4B). At 10+ months of age after these courses of therapy, 60% and 100% of the treated mice were alive, respectively, compared with 40% of those that received no therapy (FIG. 4A). These findings demonstrate that a brief blockade of the B7/CD28 costimulation pathways in combination with Rapamycin treatment were of a therapeutic benefit that continued well after treatment had been discontinued in this model of lupus.

### Example 4

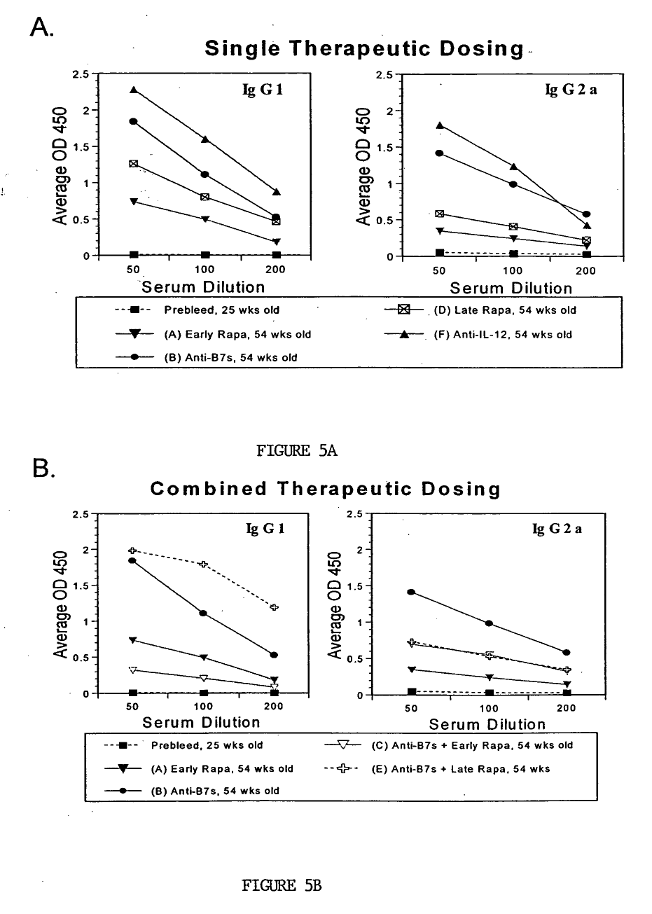

Assessment of anti-nDNA antibody isotypes were measured by ELISA and classified as either IgG₁ or IgG₂. Animals that were 25 weeks old were used as controls due to their very low circulating levels of anti-nDNA antibodies. Rapamycin was shown to be protective in 54-week old mice as evidenced by decreased production of anti-nDNA antibodies (FIG. 5A and 5B). Delayed dosing of Rapamycin did not appear to inhibit the antibody response as well as the early dosing regimen (FIG. 5A and 5B), however, this difference was not observed in the survival rates of these animals (FIG. 4A).

Equivalents

Those skilled in the art will recognize, or be able to ascertain using no more than routine experimentation, many equivalents to the specific embodiments of the invention described herein. Such equivalents are intended to be encompassed by the following claims.

1. A method for downmodulating an immune response comprising contacting immune cells from a subject with at least one antibody that binds to at least one B7 molecule in combination with a Rapamycin compound.

2. The method of claim 1, wherein the cells are contacted with two antibodies that bind at least two different B7 molecules.

3. The method of claim 1, wherein the step of contacting is performed ex vivo.

4. The method of claim 1, wherein the step of contacting is performed in vivo.

5. A method for downmodulating an immune response in a subject having an autoimmune disorder comprising contacting immune cells from the subject with at least one antibody that binds to at least one B7 molecule in combination with a Rapamycin compound.

6. The method of claim 5, wherein the immune cells are contacted with two antibodies that bind at least two different B7 molecules.

7. The method of claim 6, wherein the autoimmune disorder is systemic lupus erythematosis.

8. A method for downmodulating an immune response in a subject suffering from systemic lupus erythematosis comprising administering to the subject an antibody that binds to B7-1, an antibody that binds to B7-2, and a Rapamycin compound, wherein the antibody that binds to B7-1 and the antibody that binds to B7-2 are administered over at least one short course of therapy.

9. A method for downmodulating an immune response in a subject suffering from systemic lupus erythematosis comprising administering to the subject an antibody that binds to B7-1, an antibody that binds to B7-2, and a Rapamycin compound, wherein the Rapamycin compound is administered over at least one intermediate course of therapy.

10. A method for downmodulating an immune response in a subject suffering from systemic lupus erythematosis comprising administering to said subject an antibody that binds B7-1, an antibody that binds B7-2, and a Rapamycin compound, wherein said Rapamycin compound is administered over at least one extended course of therapy.

11. A method for downmodulating an immune response in a subject suffering from systemic lupus erythematosis comprising administering to said subject an antibody that binds B7-1, an antibody that binds B7-2, and a Rapamycin compound, wherein said Rapamycin compound is administered according to an early dosing regimen.

12. A method for downmodulating an immune response in a subject suffering from systemic lupus erythematosis comprising administering to said subject an antibody that binds B7-1, an antibody that binds B7-2, and a Rapamycin compound, wherein said Rapamycin compound is administered according to a late dosing regimen.

13. A method for downmodulating an immune response in a subject suffering from systemic lupus erythematosis comprising administering to said subject an antibody that binds B7-1, an antibody that binds B7-2, a Rapamycin compound, and an immunosupressing agents, wherein said immunosupressing agent is selected from the group consisting of FK506, Cyclosporin A and cyclophosphamide.
